(19) United States
(12) Patent Application Publication
(10) Pub. No.: US 2007/0135832 A1
(43) Pub. Date: Jun. 14, 2007

(54) VASCULAR CATHETER WITH ASPIRATION CAPABILITIES AND EXPANDED DISTAL TIP

(76) Inventors: Michael H. Wholey, San Antonio, TX (US); Mark H. Wholey, Pittsburgh, PA (US)

Correspondence Address: PIETRAGALLO, BOSICK & GORDON LLP, ONE OXFORD CENTRE, 38TH FLOOR, 301 GRANT STREET, PITTSBURGH, PA, 15219-6404, US

(21) Appl. No.: 11/566,473

(22) Filed: Dec. 4, 2006

Related U.S. Application Data
(63) Continuation-in-part of application No. 10/348,137, filed on Jan. 21, 2003.
(60) Provisional application No. 60/741,215, filed on Dec. 2, 2005.
(60) Provisional application No. 60/363,310, filed on Mar. 12, 2002.

(51) Int. Cl. A61M 29/00

(52) U.S. Cl. 606/200

(57) ABSTRACT

A vascular catheter and method of use of the catheter for aiding in balloon angioplasty and stent placement procedures are disclosed. The vascular catheter has a retrieval catheter with an expanded distal portion. The expanded distal portion has aspiration holes which allow aspiration of embolic debris during recovery of the embolic filter. A guide sheath may be used to guide the retrieval catheter past a stent and into position for aspiration and retrieval of the filter after stent placement.

### CROSS REFERENCE TO RELATED APPLICATION

This application claims the benefit of U.S. Provisional Patent Ser. No. 60/741,215 filed Dec. 2, 2005. This application is a continuation-in-part application of U.S. patent application Ser. No. 10/348,137 filed Jan. 21, 2003, which claims the benefit of U.S. Provisional Patent Application Ser. No. 60/363,310 filed Mar. 12, 2002. All of these applications are herein incorporated by reference.

### FIELD OF THE INVENTION

The present invention relates to a vascular catheter and more particularly relates to a vascular catheter for providing recovery of distal protection devices and aspiration of embolic debris created during an intervention of blood vessels.

### BACKGROUND INFORMATION

It is common practice today to open occluded or stenotic blood vessels by inserting a guide wire and then a catheter carrying a balloon shaped segment and inflating the balloon, which exerts a radial force to press stenosis outward against the wall of the blood vessel. This procedure is called balloon angioplasty. Frequently, an implantable metallic stent will also be used to provide greater strength at the stenotic portion of the blood vessel, and to provide longer-term patency.

In order to help deliver balloon catheters and stent devices, special guiding catheters or sheaths are often used. These guiding catheters or sheaths are placed upstream from the targeted lesion or stenotic area. A guide wire may be advanced past the stenotic area, allowing the subsequent balloon catheters and stents to be advanced through the guiding catheter or sheath to the target area of the blood vessel.

During a balloon angioplasty procedure and stent placement at the stenotic lesion, there may exist the risk of dislodging fragments of plaque, thrombus and/or other material. These fragments may become dislodged from the stenotic lesion when the balloon segment is inflated or during other parts of the procedure. If the lesion involves arterial circulation, then the dislodged particles could flow into smaller vessels in the brain, other organs, or extremities, thus resulting in disastrous complications. Likewise, if the lesions involve the venous circulation, then the dislodged fragments could flow into the heart and lungs, possibly resulting in the demise of the patient.

Embolic protection devices are commonly used to provide protection from such dislodged fragments of plaque and thrombus. These protection devices often consist of a small umbrella-like filter or lasso-shaped device attached to the end of a guide wire. The filter is encompassed in a delivery sheath which is advanced past the lesion into a vessel segment upstream or distal to the lesion. The delivery sheath is then pulled back exposing the filter and allowing it to expand. The sheath is then removed. While expanded, the filter can capture dislodged particles while still allowing blood to freely flow past it. The filter will usually stay expanded during all major parts of the procedure. Predilatation, which is conventionally the first step of the procedure, provides an important stent by dilating the lesion usually with a small balloon catheter (2-4 mm in diameter).

After the predilatation, a self-expandable stent is deployed and post dilated with a separate and larger balloon catheter. Embolic material created during predilatation and stent placement will be captured by the filter. However, if the debris is of a large volume, it can occlude flow through the filter. This can be a dangerous situation when the filter is recaptured, the debris could become dislodged and flow distally. When the procedure is completed, a separate retrieval catheter is often required to advance through the stented artery and used to collapse and retrieve the embolic protection device.

The separate step of inserting a separate retrieval catheter is a major disadvantage of the current system of filter recovery. In carotid stent placement, longer procedure time and more steps are directly related to increased chance of a major complication occurring. Another disadvantage of the current system is that it is frequently difficult to advance the filter and the sheath together past tight and tortuous angles of some blood vessels.

A need exists for a catheter capable of providing recovery of the deployed embolic filter while also providing a means to aspirate debris proximal to the filter and stenotic lesion of a blood vessel. A need also exists for a way of clearing a blocked embolic filter without requiring installation of a separate aspiration catheter.

The present invention has been developed in view of the foregoing, and to address other deficiencies in the prior art.

### SUMMARY OF THE INVENTION

The invention relates to an apparatus and method for providing recovery of distal embolic protection devices while at the same time providing a means of aspirating thromboembolic debris.

An aspect of the invention is to provide a vascular catheter comprising a shaft including an expanded distal tip structured and arranged to receive at least a portion of an embolic filter and a plurality of aspiration holes positioned along the shaft adjacent the expanded distal tip structured and arranged to allow aspiration of embolic material adjacent the distal tip within a blood vessel.

Another aspect of the present invention is to provide a vascular catheter comprising a shaft including an expanded distal tip structured and arranged to receive at least a portion of an embolic filter, and means for aspirating embolic material through a side of the expanded distal tip.

A further aspect of the present invention is to provide a vascular catheter having a distal end and a proximal end comprising a shaft including an expanded distal tip structured and arranged to receive at least a portion of an embolic filter, a guide sheath coaxially located inside of the shaft, a guide wire coaxially positioned within the guide sheath, and an embolic filter attached to the guide wire at a distal end, wherein the shaft and guide sheath are slidably related and the guide wire and guide sheath are slidably related.

Another aspect of the present invention is to provide a vascular catheter having a distal end and a proximal end comprising a shaft including an expanded distal tip structured and arranged to receive at least a portion of an embolic filter, a guide wire coaxially positioned within the shaft, means for guiding the shaft along the guide wire, and an embolic filter attached to the guide wire at a distal end, wherein the shaft the guide wire are slidably related.

A further aspect of the present invention is to provide s method of retrieving an embolic filter and aspirating embolic material from a blood vessel comprising retracting the embolic filter into an expanded distal tip of a retrieval catheter positioned in the blood vessel, and aspirating embolic material from the blood vessel through aspiration holes provided adjacent the expanded distal tip of the retrieval catheter.

Another aspect of the present invention is to provide a method of using a vascular catheter having an outer retrieval catheter with an expanded distal tip and a guide sheath coaxially located in the retrieval catheter comprising the steps of advancing the vascular catheter along a guide wire coaxially located within the guide sheath, the guide wire having a distally located embolic filter, and coaxially located within the guide sheath to a point where the expanded distal tip is past a stenotic lesion in a blood vessel, and retracting the embolic filter into the expanded distal tip of the retrieval catheter.

These and other aspects of the present invention will be more apparent from the following description.

### BRIEF DESCRIPTION OF THE DRAWINGS

FIG. 1 is a partially schematic longitudinal anatomical view of a catheter apparatus in accordance with an embodiment of the present invention in a representation of a human carotid artery.

FIGS. 2a and 2b are longitudinal side views of the apparatus of FIG. 1, including distal and proximal segments.

FIGS. 3a and 3b are longitudinal cross sectional views of the distal and proximal segments.

FIG. 4a is a longitudinal cross sectional view of an embodiment of the invention illustrating a distal segment with the guide sheath shortened and incorporated into the retrieval catheter. FIG. 4b is a longitudinal cross sectional view of another embodiment of the invention illustrating a distal segment with the guide sheath attached to the retrieval catheter.

FIGS. 5a and 5b are longitudinal cross sectional views of the distal and proximal segments with the guide sheath removed.

FIGS. 6a and 6b are longitudinal side views of the apparatus of FIG. 1, including distal and proximal segments with the “Y-adaptor” removed and the guide sheath attached directly to the proximal hub.

FIGS. 7a and 7b are longitudinal side views of a variation of the apparatus of FIG. 1, including distal and proximal segments with the filter guide wire exiting a sidehole.

FIGS. 8a and 8b are longitudinal cross sectional views of the distal and proximal segments of the apparatus of FIG. 6.

FIGS. 9a and 9b are longitudinal side views of a variation of another embodiment of the present invention including distal and proximal segments with the filter guide wire exiting a sidehole with a separate attached tube for aspiration.

FIGS. 10a and 10b are longitudinal cross sectional view of the distal and proximal segments of the apparatus of FIG. 8.

FIGS. 11a and 11b are longitudinal side views of an embodiment of the present invention including distal and proximal segments with the filter guide wire exiting a sidehole and the aspiration tube surrounding the main tube.

FIGS. 12a and 12b are longitudinal cross sectional views of the distal and proximal segments of the apparatus of FIG. 10.

### DETAILED DESCRIPTION

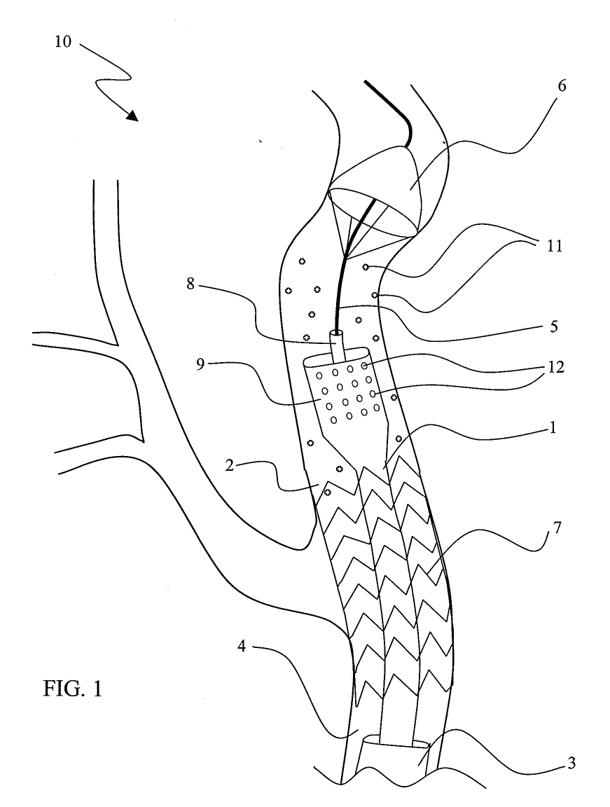

Referring now to FIG. 1, in one embodiment of the invention the device is illustrated in the human internal carotid artery 2. A guide wire 5 and embolic filter 6 are inserted into the carotid artery 4 where the embolic filter 6 is deployed at a position downstream from a lesion in the blood vessel 10. The arrows in FIG. 1 indicate the direction of blood flow. Balloon angioplasty and stent procedures may be performed once the embolic filter 6 is in place. A retrieval catheter 1, which may be a shaft with an expanded distal tip 9, is shown exiting a guide catheter 3 which is located in the common carotid artery 4. The retrieval catheter 1 is coaxially located around a guide sheath 8, which may be a flexible shaft coaxially located around the guide wire 5. After a carotid artery stent 7 has been deployed within the carotid artery 2, 4 and the stent has been post-dilated, the retrieval catheter 1 is advanced coaxially past the stent 7 with the use of the guide sheath 8. The guide sheath 8 helps to keep the expanded distal tip 9 of the retrieval catheter 1 off the edges of the stent 7. Once past the stent and in the region just proximal to the filter, the guide sheath 8 is removed. Pieces of embolic debris 11 originating from the vessel may be aspirated through aspiration holes 12 and main lumen 15 located on the expanded distal tip 9 of the retrieval catheter 1. Aspiration may be accomplished through a syringe system at the proximal end. After aspiration, the embolic filter 6 may be retracted into the expanded distal tip of the retrieval catheter. Retracting as used herein can mean pulling the filter towards the expanded distal tip 9 or advancing the retrieval catheter 1 and expanded distal tip 9 along the guide wire 5 towards the embolic filter 6 to capture the embolic filter 6. Once captured, the retrieval catheter 1 and encapsulated embolic filter 6 may be removed from the body.

In one embodiment the guide sheath 8 may be axially fixed in relation to the retrieval catheter. In this embodiment, the guide sheath 8 is filleted or slit open as the embolic filter 6 retracted into the expanded distal tip 9.

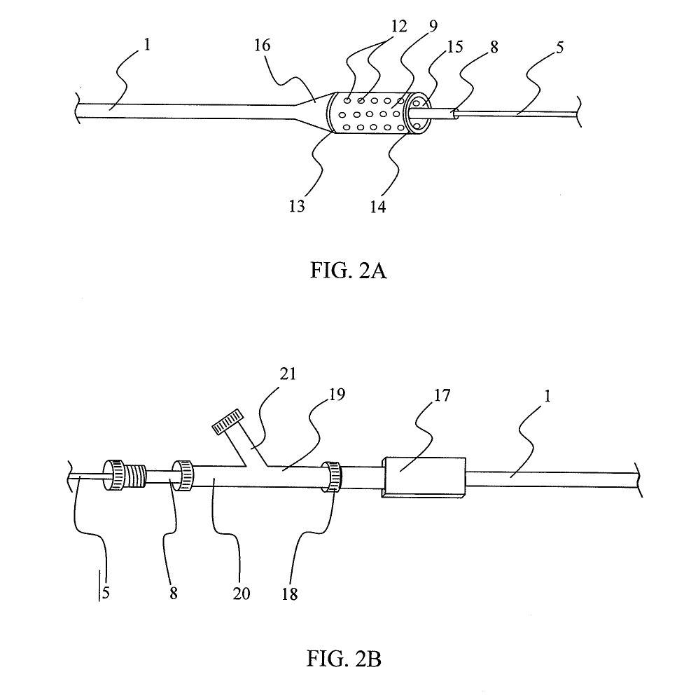

FIG. 2a shows a distal portion of the of the embodiment shown in FIG. 1 including the retrieval catheter 1 having a transition segment 16 and distal trumpeted segment 9. The transition segment 16 is the portion of the retrieval catheter wherein the diameter of the retrieval catheter 1 transitions to a greater diameter in the expanded distal tip 9. Proximal and distal radiopaque markers 13, 14 located on the expanded distal tip allow 9 the retrieval catheter 1 to be easily located. Aspiration holes 12 within the expanded distal tip 9 are used to aspirate embolic debris 11. Coaxially located between the guide wire 5 and the retrieval catheter 1 is the guide sheath 8. The interior face of the retrieval catheter is a main lumen 15. The main lumen 15 serves to aspirate embolic debris received from the aspiration holes 12 and any debris 11 received through a distal opening.

Referring now to FIG. 2b, the guide sheath 8 enters the device through the main port 20 which forms one leg of a Y-adapter 19. The other leg of the Y-adapter is the aspiration port 21. A luer end 18, or other suitable threaded or compression fitting, may connect the Y-adapter to a proximal hub 17. The distal end of the proximal hub 17 connects to the retrieval catheter 1.

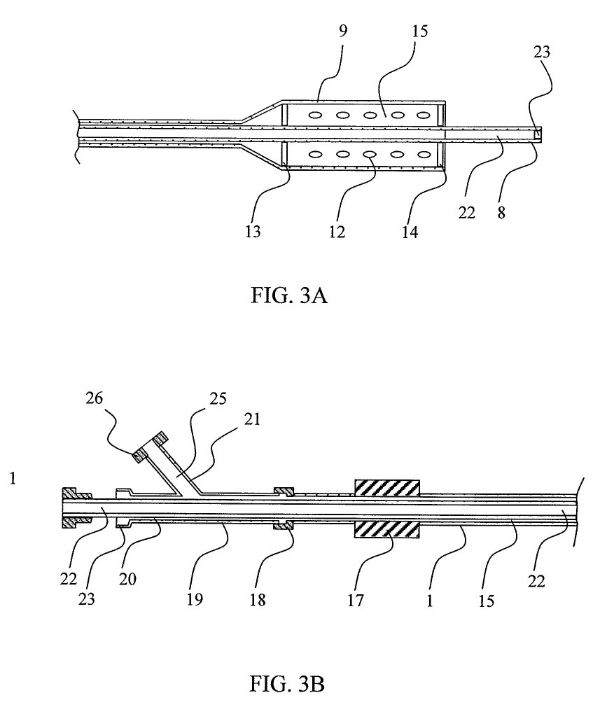

FIG. 3a shows a cross sectional view of a distal end of the retrieval catheter 1 shown in FIG. 1. The guide sheath 8 has a lumen 22 in which the filter guide wire 5 (not shown in this figure) courses coaxially. In this embodiment a distal radiopaque marker 23 is positioned at a distal end of guide sheath 8. Radiopaque markers 13, 14 are also shown at the proximal and distal ends of the expanded distal tip 9. Aspiration holes 12 are shown as seen from the interior of the expanded distal tip 9.

Referring now to FIG. 3b a cross section of the proximal end of the device of FIG. 1 is shown. Retrieval catheter 1 connects to proximal hub 17 which is then connected to Y-adapter 19 through the use of luer end 18. The guide sheath 8 and guide wire 5 (not shown) run through the retrieval catheter 1, through the proximal hub 17 and exit through the main port 20 which may also have luer end 23. Guide sheath 8 terminates at proximal end 24. The interior of aspiration port 21 forms aspiration lumen 25. Aspiration port 21 may also have luer end 26.

The embodiments shown in FIGS. 3a and 3b are typical of what a cross section of the device may look like as the retrieval catheter 1 is moved into a position past a stent 7 and close to embolic filter 6.

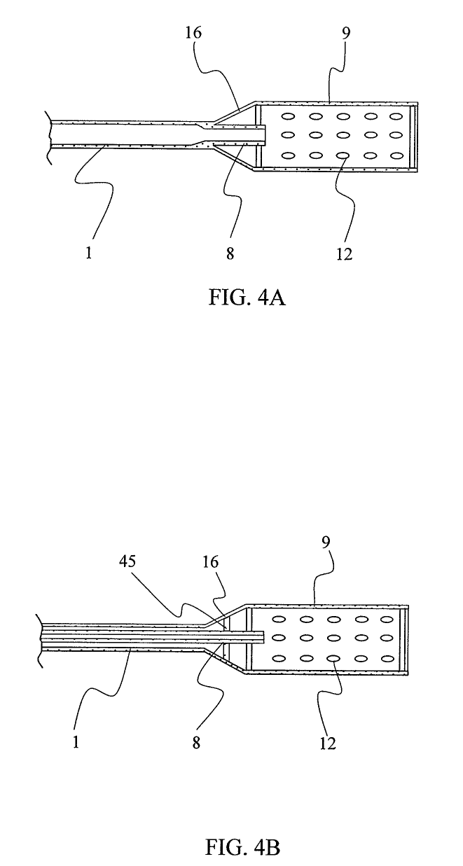

FIGS. 4a and 4b show two other embodiments of the invention. In the embodiment shown in FIG. 4a the guide sheath 8 is shortened and incorporated into the retrieval catheter 1. The guide sheath 8 continues to keep the retrieval catheter in proper alignment along the guide wire 5 (not shown) but now is fixed in relation to the retrieval catheter 1. In this embodiment, debris is aspirated through the lumen of the guide sheath 8. In the embodiment shown in FIG. 4b, the guide sheath 8 and the retrieval catheter 45 are attached together through attachment 45. As described above the guide sheath 8 keeps the retrieval catheter 1 centered on the guide wire 5 (not shown). The attachment 8 keeps the retrieval catheter 1 and guide sheath 8 axially fixed in relation to each other. The attachment 45 has perforations or spokes so that debris may still be aspirated between the guide sheath and retrieval catheter 1. The proximal end of the embodiment described in FIG. 4a or FIG. 4b may have a proximal hub 17 and Y-adapter 19 as described above or may include another proximal end as described herein.

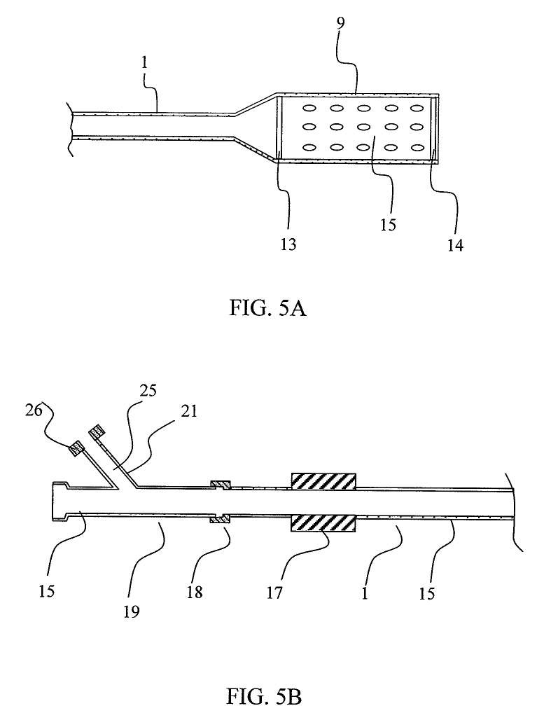

FIGS. 5a and 5b show the retrieval catheter 1 without guide sheath 8. This configuration is what the device may look like once expanded distal tip 9 is in position proximal to embolic filter 6. With guide sheath 8 removed more space is available in main lumen 15. This facilitates aspiration through aspiration port 25, main lumen 15 and aspiration holes 12. Guide wire 5 is not shown but would be present in FIGS. 5a and 5b. Connected at the distal end to an embolic filter 6 and exiting main port 20 at the proximal end.

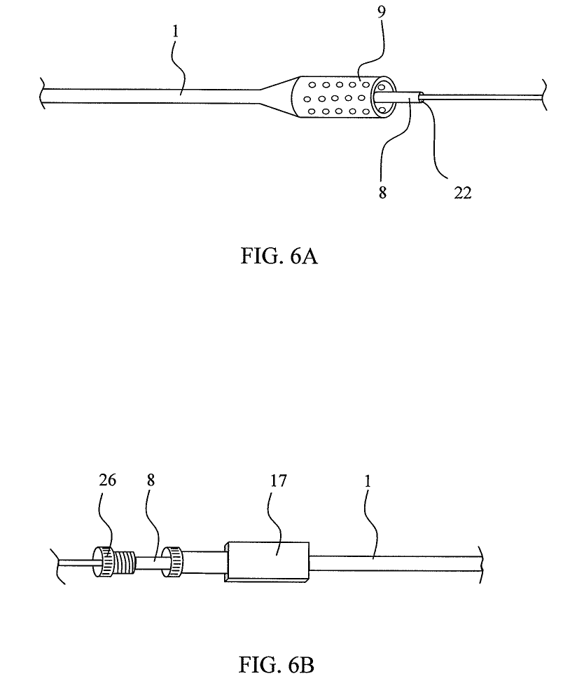

FIGS. 6a and 6b show another embodiment of the present invention in which the Y-adapter is removed. Once expanded distal tip 9 is at a position past the stent and near the embolic filter 6, the guide sheath 8 may be removed through luer end 26. Once the guide sheath 8 is removed an aspiration port (not shown) for removal of embolic debris can then be connected to luer end 26.

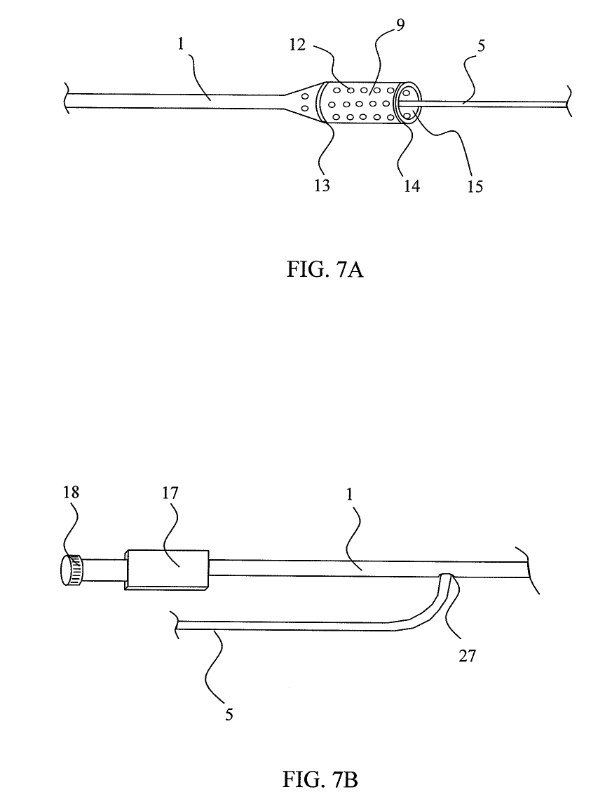

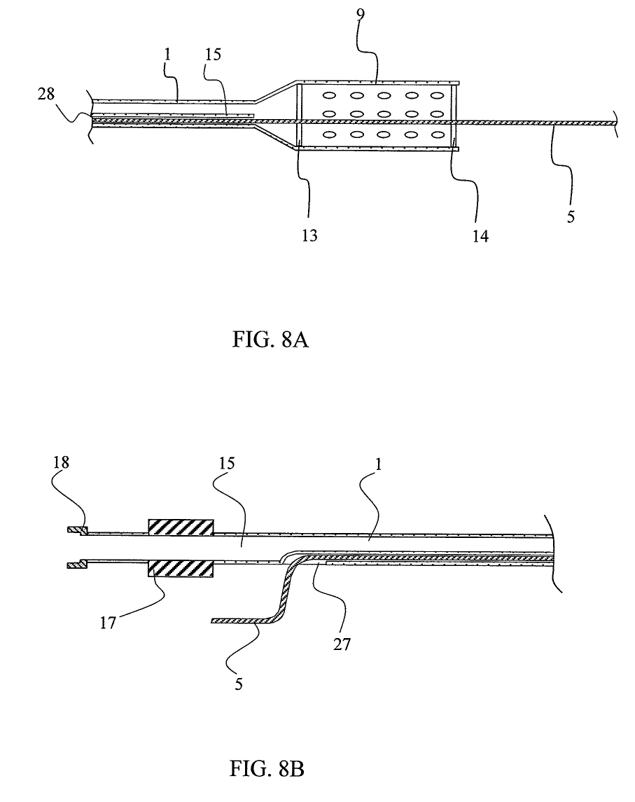

FIGS. 7 and 8 illustrate another embodiment of the present invention. A side port 27 is provided for access of the guide wire 5 through the retrieval catheter 1. A secondary lumen 28 for the guide wire 5 is shown in FIG. 8. The secondary lumen 28 is located within the main lumen 15. The main lumen 15 travels from the expanded distal tip 9 past the radiopaque markers 13, 14 and is connected to the luer end 18 through the proximal hub 17. Guide wire 5 accesses the secondary lumen 28 through a side port 27. The guide wire 5 enters the side port 27 at the proximal end of a secondary lumen 28. The guide wire 5 exits main lumen 15 of the trumpeted distal end 9 and is attached to an embolic filter 6 (not shown).

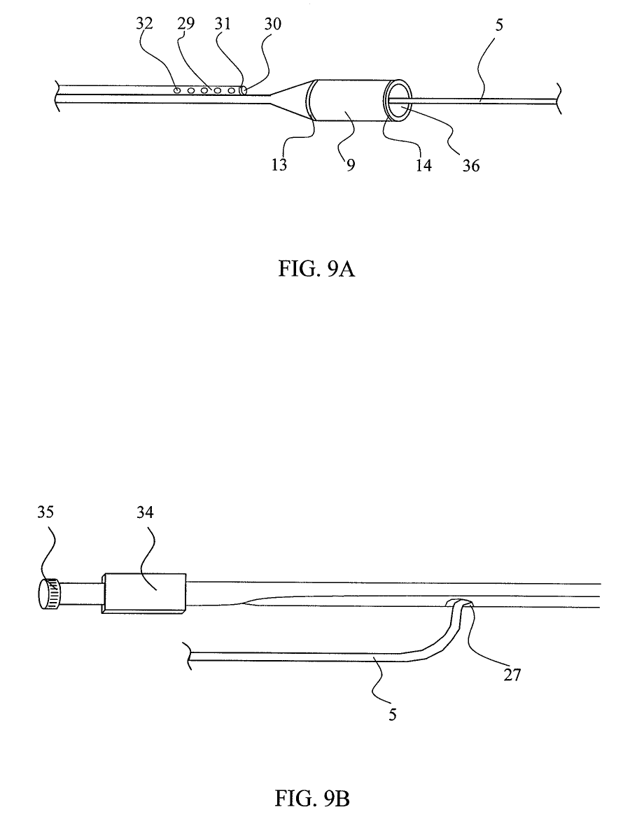

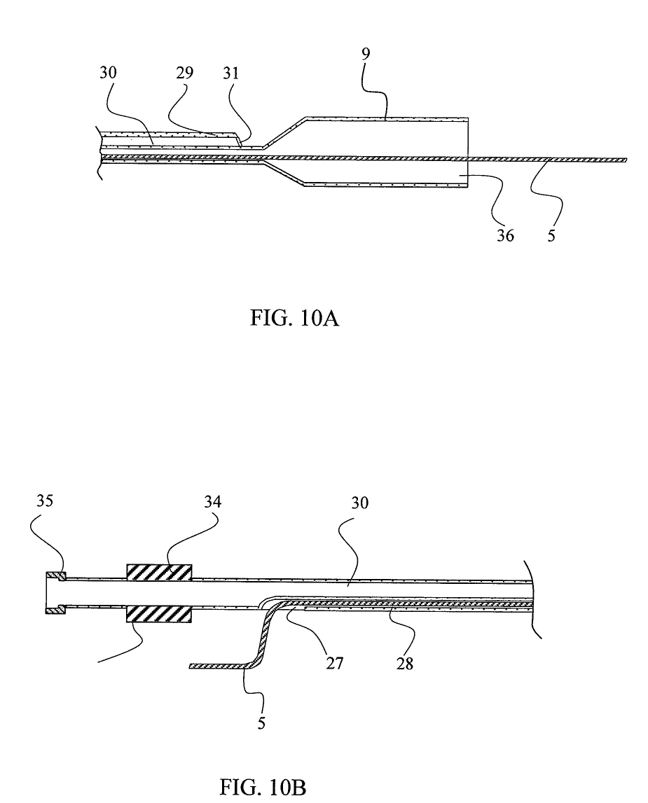

FIGS. 9 and 10 show another embodiment of the present invention wherein a separate distal aspiration tip 29, aspiration lumen 30, radiopaque marker 31 and aspiration holes 32 are provided. In this embodiment the secondary lumen 28 and side port 27 are connected but external to the aspiration lumen 30. The aspiration lumen 30 communicates with a proximal hub 34 and luer end 35 at the proximal end. At the distal end, aspiration lumen 30 may include a expanded distal tip 9 and radiopaque markers 13, 14. The guide wire 5 enters a side port 27 at the proximal end of a secondary lumen 28. The guide wire 5 exits distal end opening 36 of the trumpeted distal end 9. The guide wire 5 is attached to an embolic filter 6 (not shown).

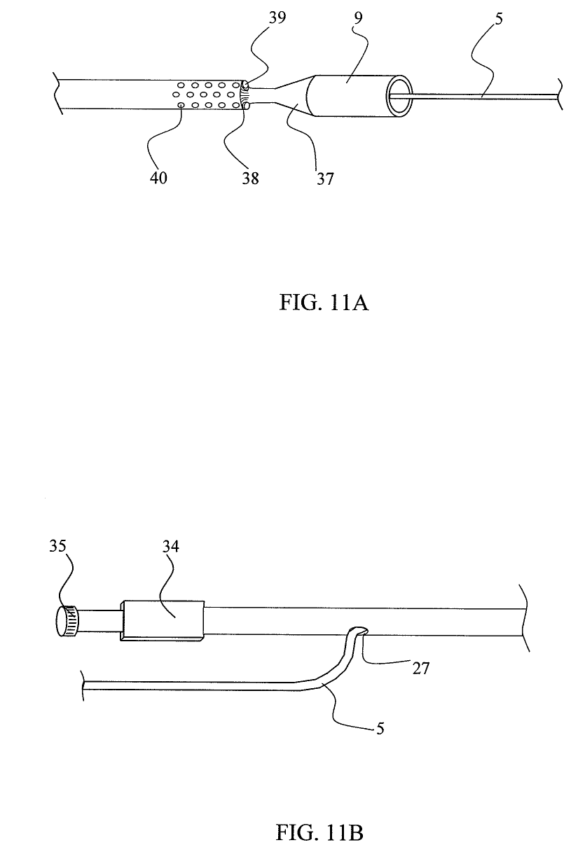

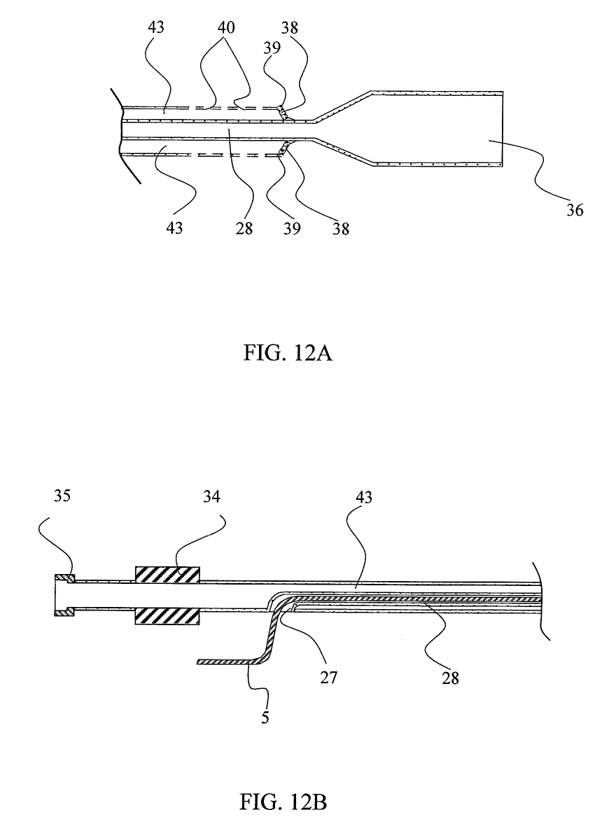

FIGS. 11 and 12 illustrate another embodiment of the present invention including a distal opening 36 of the expanded distal tip 9 of the retrieval catheter 1. In this embodiment the aspiration lumen 43 encircles the secondary lumen 28 which carries the guide wire 5. Guide wire 5 enters the proximal end at a side port 27. The guide wire 5 exits distal end opening 36 of the expanded distal tip 9. The guide wire 5 is attached to an embolic filter 6 (not shown). The expanded distal tip 9 has a tapered section 37. Aspiration ports 38 with radiopaque markers 39 and aspiration holes 40 are located at a point proximal to the tapered section. Aspiration ports 38 and aspiration holes 40 are in communication with an aspiration lumen 43 at the distal end. The aspiration lumen 43 communicates with the proximal hub 34 and luer 35 at the proximal end.

Whereas particular embodiments of this invention have been described above for purposes of illustration, it will be evident to those skilled in the art that numerous variations of the details of the present invention may be made without departing from the invention.

1. A vascular catheter comprising:
a shaft including an expanded distal tip structured and arranged to receive at least a portion of an embolic filter; and
a plurality of aspiration holes positioned along the shaft adjacent the expanded distal tip structured and arranged to allow aspiration of embolic material adjacent the distal tip within a blood vessel.

2. A vascular catheter according to claim 1, wherein at least some of the aspiration holes pass through the expanded distal tip.

3. A vascular catheter according to claim 1, wherein at least some of the aspiration holes are positioned at different locations around a circumference of the expanded distal tip.

4. A vascular catheter according to claim 3, wherein the aspiration holes form an array extending axially along and circumferentially around the expanded distal tip.

5. A vascular catheter according to claim 1, wherein at least some of the aspiration holes are provided in a lumen mounted on the shaft.

6. A vascular catheter according to claim 5, wherein the lumen is mounted on a side of the shaft.

7. A vascular catheter according to claim 5, wherein the lumen is mounted coaxially around the shaft.

8. A vascular catheter according to claim 1, further comprising a radiopaque marker adjacent the expanded distal tip.

9. A vascular catheter according to claim 1, further comprising a guide sheath coaxially located within the shaft and a guide wire coaxially located within the guide sheath, wherein the embolic filter is mounted adjacent a distal end of the guidewire.

10. A vascular catheter according to claim 1, further comprising a guide sheath coaxially located within and the shaft affixed to facilitate entry and collapse of the embolic filter.

11. A vascular catheter comprising:
a shaft including an expanded distal tip structured and arranged to receive at least a portion of an embolic filter; and
means for aspirating embolic material through a side of the expanded distal tip.

12. A vascular catheter according to claim 11, wherein the means for aspirating embolic debris comprises aspiration holes passing through the expanded distal tip.

13. A vascular catheter according to claim 12, wherein at least some of the aspiration holes are positioned at different locations around a circumference of the expanded distal tip.

14. A vascular catheter according to claim 12, wherein the aspiration holes form an array extending axially along and circumferentially around the expanded distal tip.

15. A vascular catheter according to claim 12, wherein at least some of the aspiration holes are provided in a lumen mounted on the shaft.

16. A vascular catheter according to claim 15, wherein the lumen is mounted on a side of the shaft.

17. A vascular catheter according to claim 15, wherein the lumen is mounted coaxially around the shaft.

18. A vascular catheter according to claim 11, further comprising a radiopaque marker adjacent the expanded distal tip.

19. A vascular catheter according to claim 11, further comprising a guide sheath coaxially located within the shaft and a guide wire coaxially located within the guide sheath, wherein the embolic filter is mounted adjacent a distal end of the guidewire.

20. A vascular catheter having a distal end and a proximal end comprising:
a shaft including an expanded distal tip structured and arranged to receive at least a portion of an embolic filter;
a guide sheath coaxially located inside of the shaft;
a guide wire coaxially positioned within the guide sheath; and
an embolic filter attached to the guide wire at a distal end.

21. The vascular catheter of claim 20, wherein the shaft and guide sheath are slidably related and the guide wire and guide sheath are slidably related.

22. The vascular catheter of claim 20, wherein the shaft and guide sheath are affixed.

23. The vascular catheter of claim 20, wherein the guide sheath is affixed to the shaft and has a distal end inside the expanded distal tip.

24. A vascular catheter according to claim 23, wherein the embolic filter is structured and arranged to retract into the expanded distal tip adjacent the distal end of the guide sheath.

25. A vascular catheter according to claim 20, wherein the expanded distal tip has at least one aspiration hole.

26. A vascular catheter according to claim 20, wherein a hub connects the shaft to a Y-adapter having two legs at the proximal end of the catheter.

27. A vascular catheter according to claim 26, wherein a first leg of the Y-adapter is a main port through which the guide wire and guide sheath exit at the proximal end, and wherein a second leg of the Y-adapter is an aspiration port.

28. A vascular catheter according to claim 20, further comprising a radiopaque marker adjacent the expanded distal tip of the shaft.

29. A vascular catheter according to claim 20, further comprising a radiopaque marker adjacent a distal end of the guide sheath.

30. A vascular catheter having a distal end and a proximal end comprising:
a shaft including an expanded distal tip structured and arranged to receive at least a portion of an embolic filter;
a guide wire coaxially positioned within the shaft;
means for guiding the shaft along the guide wire; and
an embolic filter attached to the guide wire at a distal end,
wherein the shaft the guide wire are slidably related.

31. A vascular catheter according to claim 30, wherein the means for guiding the shaft along the guide wire comprises a guide sheath.

32. A vascular catheter according to claim 31, wherein the guide sheath is affixed to the shaft and has a distal end inside the expanded distal tip, and the embolic filter is structured and arranged to retract into the expanded distal tip adjacent the distal end of the guide sheath.

33. A vascular catheter according to claim 30, wherein the expanded distal tip has at least one aspiration hole.

34. A vascular catheter according to claim 30, wherein a hub connects the shaft to a Y-adapter having two legs at the proximal end of the catheter.

35. A vascular catheter according to claim 30, wherein a first leg of the Y-adapter is a main port through which the guide wire and guide sheath exit at the proximal end, and wherein a second leg of the Y-adapter is an aspiration port.

36. A vascular catheter according to claim 30, further comprising a radiopaque marker adjacent the expanded distal tip of the shaft.

37. A vascular catheter according to claim 31, further comprising a radiopaque marker adjacent a distal end of the guide sheath.

38. A method of retrieving an embolic filter and aspirating embolic material from a blood vessel comprising:
retracting the embolic filter into an expanded distal tip of a retrieval catheter positioned in the blood vessel; and
aspirating embolic material from the blood vessel through aspiration holes provided adjacent the expanded distal tip of the retrieval catheter.

39. The method of retrieving an embolic filter of claim 38, further comprising the step of retracting the retrieval catheter from the blood vessel with the retracted embolic filter at least partially inside of the expanded distal tip after aspirating the embolic material.

40. The method of retrieving an embolic filter of claim 38, wherein the step of aspirating embolic material occurs prior to the step of retracting the embolic filter.

41. A method of using a vascular catheter having an outer retrieval catheter with an expanded distal tip and a guide sheath coaxially located in the retrieval catheter comprising the steps of:
advancing the vascular catheter along a guide wire coaxially located within the guide sheath, the guide wire having a distally located embolic filter, and coaxially located within the guide sheath to a point where the expanded distal tip is past a stenotic lesion in a blood vessel; and
retracting the embolic filter into the expanded distal tip of the retrieval catheter.

42. The method of using a vascular catheter of claim 41, further comprising retracting the retrieval catheter, guide wire and embolic filter from the blood vessel.

43. The method of using a vascular catheter of claim 41, further comprising the step of retracting the guide sheath from the blood vessel after inserting the vascular catheter.

44. The method of using a vascular catheter of claim 41, wherein the expanded distal tip of the retrieval catheter comprises aspiration holes.

45. The method of using a vascular catheter of claim 41, further comprising the step of aspirating embolic debris from an area between the embolic filter and stenotic lesion.

46. The method of using a vascular catheter of claim 45, wherein the aspirating is conducted through aspiration holes provided in the expanded distal tip of the retrieval catheter before, during or after retracting the embolic filter.

47. The method of using a vascular catheter of claim 41, wherein the guide sheath is inserted in the blood vessel before the retrieval catheter.

48. The method of using a vascular catheter of claim 41, further comprising the steps of:
inserting a guide catheter into a blood vessel;
inserting a guide wire with an attached and undeployed embolic filter through the guide catheter and past the stenotic lesion in the blood vessel;
deploying the embolic filter; and
performing balloon angioplasty or stent placement in the blood vessel before advancing the vascular catheter.
